(19) United States
(12) Patent Application Publication
(10) Pub. No.: US 2004/0063100 A1
(43) Pub. Date: Apr. 1, 2004

(54) Nanoneedle chips and the production thereof

(76) Inventor: Chung Lin Wang, Kaohsiung (TW)

Correspondence Address: HARNESS, DICKEY & PIERCE, P.L.C., P.O. Box 8910, Reston, VA, 20195, US

(21) Appl. No.: 10/259,849

(22) Filed: Sep. 30, 2002

(51) Int. Cl. C12Q 1/68; C12M 1/34; H01L 21/00

(52) U.S. Cl. 435/6; 435/287.2; 438/1

(57) ABSTRACT

The invention relates to a nanoneedle chip, which comprises a support having an interface region; and a plurality of shafts connected to and extending from said interface region of the solid support, wherein each said shaft is in a cone-like or cylinder shape, the tip of each shaft ranges from about 0.1 nm to less than about 1 μm in diameter, the base of each shaft ranges from about 1 nm to less than about 1 μm in diameter and the height of shaft is at least three times than the diameter of the base. Also disclosed in the processes for producing the nanoneedle array of the invention.

### BACKGROUND OF THE INVENTION

1. Field of the Invention

The present invention relates to a new nanoneedle chip and the production thereof.

2. Description of the Prior Art

Microinjection techniques have been used in biological research for a number of applications that include protein and pathogen injection, high efficiency transformation, organelle transfer, genetic material delivery and transgenic techniques. The needles in micro-scale have the potential to replace standard syringes in applications. Because of the small dimension of the microneedles, they can be inserted into the body painlessly and cause less tissue damage than general needles. Therefore, microneedles have been developed and applied in the microinjection techniques. For example, U.S. Pat. No. 6,090,790 provided a microneedle for delivering genetic materials into a target cell site. U.S. Pat. No. 6,331,266 disclosed a method of manufacturing a micro-device. Moreover, microneedle array systems are developed to increase the injection efficiency. U.S. Pat. No. 6,379,324 disclosed a hollow microneedle array constructed of silicon dioxide compounds using Micro-Electro-Mechanical Systems (MEMS) technology and standard microfabrication techniques. Boris Storeber and Dorian Liepmann taught out-of-plane hollow microneedles for injecting insulin and other therapeutic agents (Poster 34, pp. 224-226). McAllister et al. provided three-dimensional arrays of hollow microneedles and microtubes that are fabricated from both silicon and electrodeposited metals (D. V. McAllister, F. Cros, S. P. Davis, L. M. Matta, M. R. Prausnitz, M. G. Allen, “Three-Dimensional Hollow Microneedle and Microtube arrays,” the 10th International Conference on Solid-State Sensors and Actuators, Sendai, Japan, 1999.

Because the size of cells is small, the microneedles may destroy the cells and cause the leakage of cell contents. In addition, many cells have a high internal pressure (the turgor pressure), which will worsen the problem of the leakage of cell contents. The above-mentioned problems can be reduced by making the needle as small as possible. U.S. Pat. No. 6,063,629 developed a nanopipette whose tip has a diameter of 0.025 μm to 0.3 μm. However, the nanopipette of U.S. Pat. No. 6,063,629 is merely a single pipette rather than an array, and is produced by-extruding glass to form a single pipette. It is technically difficult to produce an array with the needles in nano-scale.

### SUMMARY OF THE INVENTION

The invention relates to a nanoneedle chip, which comprises:

a support having an interface region; and

a plurality of nanoshafts connected to and extending from said interface region of the solid support, wherein each said nanoshaft is in a shape of cone-like or cylinder, the top of each nanoshaft ranges from about 0.1 nm to less than about 1 μm in dimension, the base of each nanoshaft ranges from about 1 nm to less than about 1 μm in dimension, and the height of each nanoshaft is at least three times the dimension of the base.

The invention also relates to a process for producing a solid nanoneedle chip, comprising the steps of:

(i) providing a solid support having an interface region;

(ii) coating a layer of a material on the interface region of the solid support to form nanoshafts wherein the material is selected from the group consisting of silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, ceramics and polymers;

(iii) coating photoresist on the layer of the material;

(iv) performing photolithography to form an array of dots;

(v) etching the material to transfer the dot patterns onto the solid support;

(vi) etching the solid support to a predetermined depth to define standing posts;

(vii) removing the photoresist and material on the standing posts; and

(viii) etching the posts by a chemical solution over a controlled time to form cone or cylinder-shaped solid nanoshafts, wherein the top of each nanoshaft ranges from about 0.1 nm to less than about 1 μm in dimension, the base of each nanoshaft ranges from about 1 nm to less than about 1 μm in dimension, and the height of each nanoshaft is at least three times the dimension of the base.

The invention further relates to a process for producing a hollow nanoneedle chip, comprising the steps of:

(i) providing a solid support having an interface region;

(ii) coating a layer of a material to form nanoshafts on the interface region of the solid support, wherein the material is selected from the group consisting of silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, ceramics and polymers;

(iii) coating photoresist on the layer of the material;

(iv) performing photolithography to form an array of dots;

(v) etching the material to transfer the dot patterns onto the solid support;

(vi) etching solid support to a predetermined depth to define the standing posts;

(vii) removing the photoresist and material on the posts;

(viii) growing a conformal film by atomic layer chemical vapor deposition, ultra high vacuum chemical vapor deposition, and displacement deposition to cover the whole solid support;

(ix) removing the upper layer of the conformal film by chemical mechanical polishing; and

(x) etching the solid support to form the hollow support and nanoshafts wherein the nanoshafts are in a shape of cone-like or cylinder and the top of each nanoshaft ranges from about 0.1 nm to less than about 1 μm in dimension, the base of each nanoshaft ranges from about 1 nm to less than about 1 μm in dimension, and the height of each nanoshaft is at least three times the dimension of the base.

### BRIEF DESCRIPTION OF THE DRAWINGS

The following is a brief description of the drawings, in which:

FIG. 1 is a cross-section view of the nanoneedle chip of solid nanoshafts.

FIG. 2 is an elevational view in cross-section of the nanoneedle chip of the invention having hollow nanoshafts.

FIG. 3 is a schematic of producing the nanoneedle chip of solid nanoshafts.

FIG. 4 is a schematic of producing the nanoneedle chip of hollow nanoshafts.

FIG. 5 is a scanning electron micrograph of the nanoneedle chip of solid nanoshafts. FIGS. 5a and 5b show that the top of the shafts is about 1 angstrom in dimension and the base of the shafts is 1 μm; FIG. 5c shows that the top of the nanoshafts is 0.6 μm in dimension and the base of the nanoshafts is 1 μm in dimension; FIG. 5d shows that the top of the nanoshafts is 0.2 μm in dimension and the base of the nanoshafts is 1 μm in dimension.

FIG. 6 is a chemiluminescence picture showing that the hybridization of poly dA with poly dT.

### DETAILED DESCRIPTION OF THE INVENTION

The invention provides a new nanoneedle chip and the production process thereof. The nanoneedle chip of the invention can be used in various biological applications such as in the delivery or collection of samples.

Nanoneedle Chip

The invention relates to a nanoneedle chip, said chip comprises:

a support having an interface region; and

a plurality of nanoshafts connected to and extending from said interface region of the solid support, wherein each said nanoshaft is in a shape of cone-like or cylinder, the top of each nanoshaft ranges from about 0.1 nm to less than about 1 μm in dimension, the base of each nanoshaft ranges from about 1 nm to less than about 1 μm in dimension, and the height of each nanoshaft is at least three times the dimension of the base.

According to the invention, the support of the nanoneedle chip may be hollow or solid. According to the invention, for a solid support of the nanoneedle chip, the support can be constructed from a variety of materials including Si, GaAs, III-V semiconductor compounds, metals, ceramics or glass. Preferably, the support is constructed from Si, III-V semiconductor compounds or glass. More preferably, the support is constructed from Si or glass. Most preferably, the support is constructed from Si. According to the invention, for a hollow support of the nanoneedle chip, the support can be constructed from a variety of materials including amorphous silicon, polysilicon dioxide, dry silicon dioxide, tetraehtylorthosilicate, silicon oxynitride, silicon carbide, gallium arsenic, aluminum oxide titanate, lead zirconium tantalite, metal and metal oxide. The support includes the interface region to which the nanoshafts are integrally formed.

According to the invention, the nanoshafts of the chip are formed from the interface region of the support. Each nanoshaft is in a shape of cone-like or cylinder. The nanoshafts can be constructed from a variety of materials, including silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, ceramics and polymers. More preferably, the material is selected from the group consisting of wet silicon oxide, dry silicon oxide, chemical silicon oxide, silicon nitride, silicon carbide and silicide.

According to the invention, the top of the nanoshafts ranges from about 0.1 nm to less than about 1 μm in dimension and the base of the nanoshafts ranges from about 1 nm to less than about 1 μm in diameter. Preferably, the top of the nanoshafts ranges from about 0.1 m to 0.5 nm in dimension and the base of the nanoshaft ranges from about 3 nm to 1 μm in dimension. According to the invention, the height of the nanoshafts is at least three times the dimension of the base. Preferably, the height of the nanoshafts is six to twelve times the dimension of the base of the nanoshafts. According to the invention, a nanoneedle chip can include a mixture of nanoshafts having, for example, various height, dimension, cross-sectional shape, density and spacing. According to the invention, the nanoshafts are formed on the interface region of the solid support at a density greater than 10,000 shafts/cm².

According to the invention, the nanoshafts can also be solid or hollow. As used herein, the term “hollow” means having one or more substantially annular bores or channels through the interior of the shaft structure, having a dimension sufficiently large to permit the passage of fluid and/or solid materials through the shaft. The annular bores may extend throughout all or a portion of the nanoshaft in the direction of the top to the base, extending parallel to the direction of the nanoshaft or branching or exiting at a side of the nanoshaft, if appropriate. Persons skilled in the art can select the appropriate bore features required for specific applications. For example, one can adjust the bore dimension to permit passage of the particular material to be transported through the shaft.

According to the invention, the nanoshafts can be oriented perpendicular or at an angle to the support. Preferably, the nanoshafts are oriented perpendicular to the solid support so that a larger density of the nanoshafts per unit area of substrate can be provided. A nanoneedle chip can include a mixture of nanoshaft orientations, dimensions, density, heights, or other parameters.

According to the invention, the nanoneedle chips of the invention further comprises a device for applying the electric voltage or the electric current wherein the device is connected to the support of the chip. The chip of the invention may be specifically used for sampling or for releasing the samples by applying electric voltage or electric current to the nanoshafts.

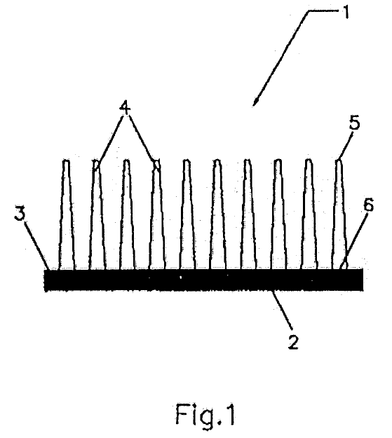

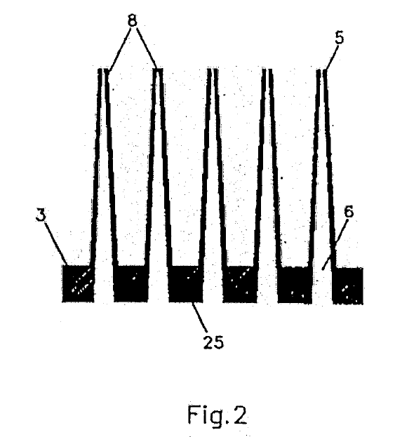

According to the preferred embodiments of the invention, the solid and hollow nanoneedle chip of the invention are illustrated in FIG. 1 and FIG. 2 respectively.

FIG. 1 depicts the solid nanoneedle chip 1 including a solid support 2 having an interface region 3 and solid nanoshafts 4. The nanoshafts include a top 5 and a base 6. The top 5 of the shafts 4 can be inserted into a target so that the samples may be delivered to or taken out of the target. For example, the samples to be delivered are coated on the top 5 of the nanoshafts and then delivered into the target by piercing the top 5 into the target.

FIG. 2 depicts the hollow nanoneedle chip 7 including a support 25 having an interface region 3 and hollow nanoshafts 8. The hollow nanoshaft may further contain a microflow channel therein along its length. The shafts 8 include a top 10 and a base 11. The top 10 of the nanoshafts 8 can pierce targets so that samples may be delivered into or taken out of the targets, for example, via a top partport. A microflow channel is connected to a microchamber for tapping the samples to be delivered. In addition, micropumps and microvalves are incorporated into the chip.

Production Process of the Nanoneedle Chip

The nanoneedle chip of the invention may be solid or hollow. The process for producing the solid nanoneedle chip is different from that for producing the hollow nanoneedle chip.

Production of solid Nanoneedle Chip

The invention relates to a process for producing a solid nanoneedle chip, comprising the step of:

(i) providing a solid support having an interface region;

(ii) coating a layer of a material on the interface region of the solid support to form nanoshafts wherein the material is selected from the group consisting of silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, ceramics and polymers;

(iii) coating photoresist on the layer of the material;

(iv) performing photolithography to form an array of dots;

(v) etching the material to transfer the dot patterns onto the solid support;

(vi) etching the solid support to a predetermined depth to define standing posts;

(vii) removing the photoresist and material on the standing posts; and

(viii) etching the posts by a chemical solution over a controlled time to form the cone or cylinder-shaped solid nanoshafts, wherein the top of each nanoshaft ranges from about 0.1 nm to less than about 1 μm in dimension, the base of each nanoshaft ranges from about 1 nm to less than about 1 μm in dimension, and the height of each nanoshaft is at least three times the dimension of the base.

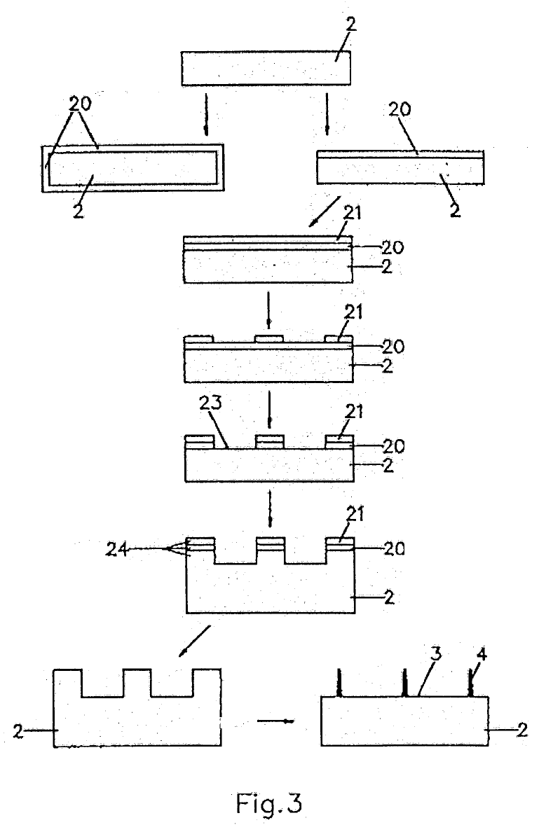

According to the embodiment of the invention, the process for producing the solid nanoneedle chip can be shown in FIG. 3. The solid support 2 has an interface region 3 wherein the shafts 4 is formed thereon. A material 20 is coated onto the inferface region of the solid support 2 to form shafts wherein the material is select from the group consisting of silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, compounds, ceramics and polymers. Preferably, the material is selected from the group consisting of wet silicon oxide, dry silicon oxide, chemical silicon oxide, silicon nitride, silicon carbide and silicide. Optionally, the material may be coated on the whole solid support of the chip.

Then, photoresist 21 is coated on the material 20. Photolithography is performed to form an array of dots. Preferably, the photolithography is performed by G-line stepper, I-line stepper, excimer laser r, E beam lithography, ion beam lithography, soft X-ray technique, or laser drilling. The material 20 is etched to transfer the dot patterns onto the solid support. The etching is performed by transformed coupled plasma etcher, inductively coupled plasma etcher, electron cyclotron resonance etcher, high density plasma etcher, reactive ion etcher or cryo reactive ion etcher. Further, the solid support without dots 23 are etched to a predetermined depth to define standing posts 24. The photoresist 21 and material 20 on the standing posts 24 are removed. Then, the posts 24 are etched by a chemical solution over a controlled time to form the nanoshafts, wherein the top of each shaft ranges from about 0.1 nm to less than about 1 μm in dimension, the base of each shaft ranges from about 1 nm to less than about 1 μm in dimension, and the height of each nanoshaft is at least three times the dimension of the base. According to the invention, the chemical solution is selected from the group consisting of a KOH, HF, and a nitric acid mixture.

Production of Hollow Nanoneedle Chip

The invention also relates to a process for producing a hollow nanoneedle chip, comprising the steps of:

(i) providing a solid support having an interface region;

(ii) coating a layer of a material to form nanoshafts on the interface region of the solid support, wherein the material is selected from the group consisting of silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, ceramics and polymers;

(iii) coating photoresist on the layer of the material;

(iv) performing photolithography to form an array of dots;

(v) etching the material to transfer the dot patterns onto the solid support;

(vi) etching solid support to a predetermined depth to define the standing posts;

(vii) removing the photoresist and material on the posts;

(viii) growing a conformal film by atomic layer chemical vapor deposition, ultra high vacuum chemical vapor deposition, and displacement deposition to cover the whole solid support;

(ix) removing the upper layer of the conformal film by chemical mechanical polishing; and

(x) etching the solid support to form the hollow support and nanoshafts wherein the nanoshafts are in a shape of cone-like or cylinder and the top of each nanoshaft ranges from about 0.1 nm to less than about 1 μm in dimension, the base of each nanoshaft ranges from about 1 nm to less than about 1 μm in dimension, and the height of each nanoshaft is at least three times the dimension of the base.

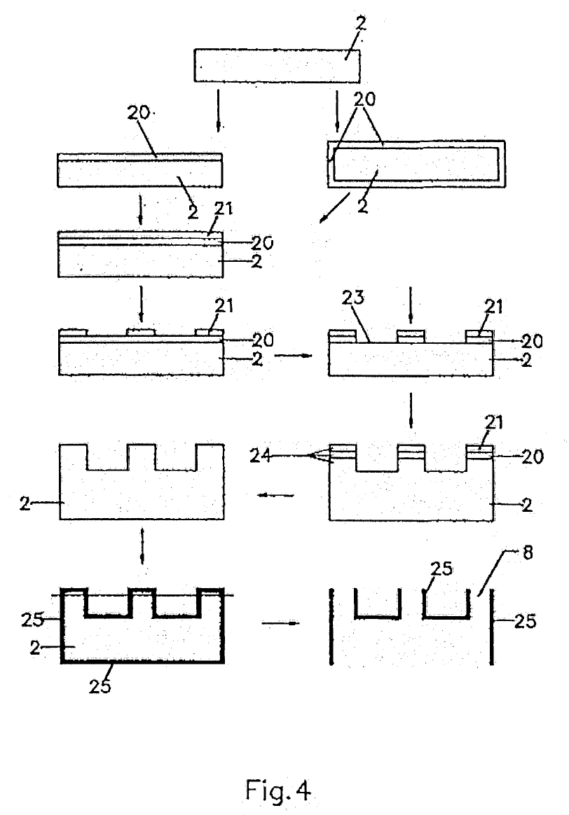

According to an embodiment of the invention, the process for producing the nanoneedle chip with hollow nanoshafts is shown in FIG. 4. The solid support 2 has an interface region 3 wherein the shafts 4 is formed thereon. A material 20 is coated onto the inferface region of the solid support 2 wherein the material is selected from the group consisting of silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, compounds, ceramics and polymers. Preferably, the material is selected from the group consisting of wet silicon oxide, dry silicon oxide, chemical silicon oxide, silicon nitride, silicon carbide and silicide. Optionally, the material may be coated on the whole solid support of the chip.

Then, photoresist 21 is coated on the material 20. Photolithography is performed to form an array of dots. Preferably, the photolithography is performed by G-line stepper, I-line stepper, excimer laser r, E beam lithography, ion beam lithography, soft X-ray technique, or laser drilling. The material 20 is etched to transfer the dot patterns onto the solid support. The etching is performed by transformed coupled plasma etcher, inductively coupled plasma etcher, electron cyclotron resonance etcher, high density plasma etcher, reactive ion etcher or cryo reactive ion etcher. Further, the solid support without dots 23 are etched to a predetermined depth to define standing posts 24. The photoresist 21 and material 20 on the standing posts 24 are removed. A conformal film 25 is grown to cover the whole solid support. According to the invention, the conformal film 25 is selected from the group consisting of wet silicon, dioxide, dry silicon dioxide, tetraethylorthosilicate, silicon nitride, silicon, oxynitride, silicon carbide, gallium arsenide, aluminum oxide, barium strontium titanate, lead zirconium tantalate, metal, metal oxide, organic material and polymer.

The upper layer of the conformal film is removed by chemical mechanical polishing. Then, the solid support 2 is removed by chemical solution to define shafts to form the hollow nanoneedles. The top of each nanoshaft ranges from about 0.1 nm to less than about 1 μm in dimension, the base of each nanoshaft ranges from about 1 nm to less than about 1 μm in dimension, and the height of each nanoshaft is at least three times the dimension of the base. According to the invention, the chemical solution is selected from the group consisting of KOH, HF, and a nitric acid mixture.

Utility

The nanoneedle chip of the invention may be used for single or multiple uses for rapid transport across a biological barrier or may be left in a place for longer time (e.g., hours or days) for the long-term transport of molecules. Depending on the dimensions of the chip, the application site, and the route in which the chip is introduced into (or onto) the biological barrier, the chip may be used to introduce or remove molecules at specific locations. The nanoneedle chip can successfully penetrate cells without causing damage either the needles or cells.

According to one embodiment of the invention, the nanoshafts of the chip of the invention can further comprise a carbon nanotube which is attached to the nanoshafts of the chip of the invention. The carbon nanotube can be used to deliver and remove samples.

Delivery of Samples

Particularly, the nanoneedle chip of the invention can be used in delivering samples to nuclei, mitochondria, chloroplasts, cells, tissues, organs, or removing samples from nuclei, mitochondria, chloroplasts, cells, tissues, organs. For example, the samples can be selected from the group consisting of drugs, DNAs, RNAs, genes, expressible genetic materials, plasmids, chromatin, chromosomes, nuclei, nucleoli, viruses, mitochondria, thylakoids, granas, chloroplasts, Golgi apparatus, endoplasmic reticulum, lysosomes, peroxisomes, centrioles, vacuoles, lipid bilayers, ribosomes, plasma membranes, cytosols, filamentous cytoskeleton, drugs, toxicants, nutritions, proteins, enzymes, substrate, cell organelles, liposomes, cells, inorganic nano particles, nano particles which are attached by the above samples. According to the invention, for the solid nanoneedle chip, the samples may be delivered by coating them on the solid nanoshafts of the chip of the invention. For the hollow nanoneedle chip, the samples may be delivered by using the hollow nanoshafts having a microflow channel therein along its length in combination with an aligned microchambers for tapping target materials to be delivered.

Removal of Samples

The nanoneedle chip of the present invention is expected to have broad applications on sample sampling and precisely located chemical-reaction stimulation. The chip of the invention may further comprise a device for applying electric voltage, or electric current to conduct the sample sampling, sample replication and chemical-reaction stimulation.

According to one preferred embodiment of the invention, the nanoneedle chip can be used in a specific sampling by immobilizing materials specific to the target samples on the nanoshafts of the chip of the invention. The materials specific to the target samples (such as DNA, RNA, antigen and antibody) can be immobilized on the suitable location of the nanoshafts of chip. The nanoshafts can be inserted into the target so that the specific samples may bind to the materials. After taking out the chip of the invention, the samples could be analyzed.

Replication of Samples

According to another embodiment of the invention, the nanoneedle chip of the invention can be used in the specific replication of samples. For example, the single strand DNAs are immobilized in the microwell plate as templates. The reactants for a polymerase chain reaction are added to the microwell plate. A PCR is performed so that the DNAs are duplicated in the microwell plate. The nanoshafts of the nanoneedle chip of the invention is connected to a device for applying electric voltage or electric current. After applying electric current to the nanoshafts of the nanoneedle chip of the invention, the nanoshafts bear charges. The resulting nanoshafts of the nanoneedle chip are put into the microwell and the antisence strands of the DNAs are binded to the nanoshafts of the chip of the invention. The nanoshafts of the chip bearing the antisence DNAs are put into another microwell. The antisence DNAs can be removed from the nanoshafts and dropped into the another microwell plate by changing the polarity of the electric voltage. By repeating the above-mentioned reaction cycle, a number of microwell plates containing the same DNAs can be obtained.

By the way of the delivery and removal of target materials, the chip of the invention may be used in gene therapy, genetic pharmacology, DNA vaccination/immunization, cancer biology, skin repair/wound healing, infectious diseases, gene expression, and detection and diagnosis of diseases.

The following Examples are offered by way of illustration and not by way of limitation.

### EXAMPLES

### Example 1

### Production of Solid Nanoneedle Chip

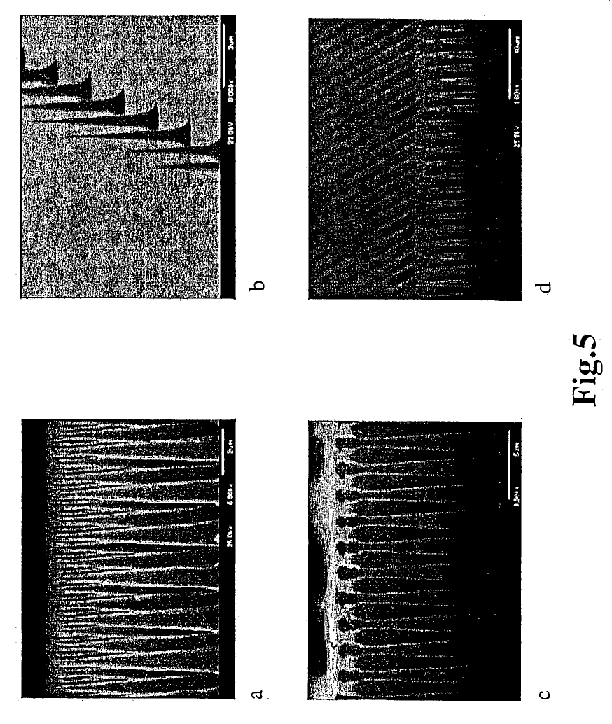

A six inches of silicon wafer was used as a solid support for the chip fabrication. Initially, the wafer was thermally oxidized at 900° C. in a steam ambient with TCA to form a 1 μm thick layer of SiO₂. The wafer was then primed at 90° C. in HMDS vapor to promote photoresist adhesion, followed by photoresist coating. A photolithography step was performed using a 10× I-line step-and-repeat system to form an array of 0.4-1 μm dots. The dot patterns were then transferred onto the silicon dioxide layer by reactive ion etching, using CHF₃ until the open areas were free of SiO₂. This patterned silicon dioxide then served as a hard mask to etch the underlying silicon to define standing posts. These posts were etched using cryo electron cyclotron resonance to obtain 10-15 μm tall silicon posts. The resulting posts were etched by nitric acid mixtures over a predetermined time to obtain the nanoshafts of the chip of the invention (see FIG. 5).

### Example 2

### Production of the Hollow Nanoneedle Chip

A six inches of silicon wafer was used as solid support for the chip fabrication. Initially, the wafer was thermally oxidized at 900° C. in a steam ambient with TCA to form a 1 μm thick layer of SiO₂. The wafer was then primed at 90° C. in HMDS vapor to promote photoresist adhesion, followed by photoresist coating. A photolithography step was performed using a 10× I-line step-and-repeat system to form an array of 0.4-1 μm dots. The dot patterns were then transferred onto the silicon dioxide layer by reactive ion etch, using CHF₃ until the open areas were free of SiO₂. This patterned silicon dioxide then served as a hard mask to etch the underlying silicon to define standing posts. These posts were etched using cryo ECR to obtain 10-15 μm tall silicon posts. The posts are coated with 0.1 μm of Al₂O₃ using atomic layer chemical vapor deposition. Remove 0.1 μm of the thickness of the conformal film from the top of the posts by chemical mechanical polishing. Photolithography was performed on the backside of the wafer using an infrared aligner. This defined windows for through-wafer etch. The wafer was then immersed in a 34 wt % of potassium hydroxide solution at 70° C. until the cores of the nanoshafts are removed to form the hollow nanoneedle chip.

### Example 3

### Delivery of plasmid by Using the Chip of the Invention

5 μl of 1 μg/μl plasmid pCMV EGFP was dropped on the chip of solid nanoneedles described in Example 1. The resulting chip was applied to the 3T3 or MCF7 cells and the cells encoding GFP were obtained.

### Example 3

### Hybridization Reaction in the Chip of the Invention

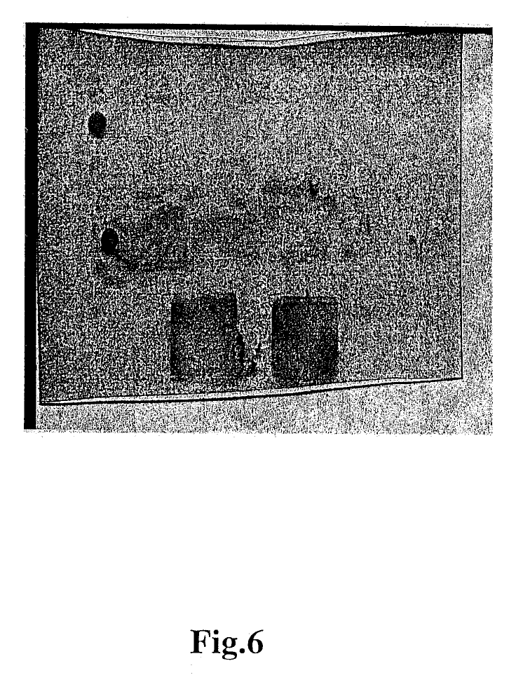

5 μl of 1 μg/μl poly dA containing sulfur group were immobilized on the solid nananeedle chip as described in Example 1, which are coated with a layer of Au. After 12 hours, the chip was washed by DDI water and then 0.2% SDS for 2 hours. The resulting chip was washed by DDI water for 2 hours. The poly dT containing biotin was dropped on the chip for hybridization. After 12 hours, the resulting chip was washed by DDI water and then 0.2% SDS for 2 hours, and then DDI water for another 2 hours. The avidin containing biotin and marked with fluorescence was added on the chip and the peroxidase was than added to detect the presence of biotin. The chemiluminescence shown in FIG. 6 indicates that the chip of the invention indeed performs a specific hybridization reaction. The black squares in the chemiluminescence picture represent the nanoneedle chip of the invention.

What is claimed is:

1. A nanoneedle chip, which comprises:
a support having an interface region; and
a plurality of nanoshafts connected to and extending from said interface region of the solid support, wherein each said shaft is in a shape of cone-like or cylinder, the top of each shaft ranges from about 0.1 nm to less than about 1 μm in dimension, the base of each shaft ranges from about 1 nm to less than about 1 μm in dimension, and the height of each nanoshaft is at least three times the dimension of the base.

2. The chip of claim 1, wherein the support is solid and the solid support is made of the material selected from the group consisting of Si, GaAs, III-V semiconductor compounds, metals, ceramics, polymer, organic materials and glass.

3. The chip of claim 1, wherein the material is Si.

4. The chip of claim 1, wherein the support is hollow and the hollow support is made of the material selected from the group consisting of amorphous silicon, polysilicon dioxide, dry silicon dioxide, tetraehtylorthosilicate, silicon oxynitride, silicon carbide, gallium arsenic, aluminum oxide titanate, lead zirconium tantalite, metal and metal oxide.

5. The chip of claim 4, wherein the material is Si.

6. The chip of claim 1, wherein the nanoshafts are made of a material selected from the group consisting of silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, ceramics and polymers.

7. The chip of Clam 1, wherein the nanoshafts are solid.

8. The chip of claim 1, wherein the nanoshafts are hollow.

9. The chip of claim 1, wherein the top of the nanoshaft ranges from about 0.1 nm to about 1 μm in dimension, the base of the nanoshaft ranges from about 3 nm to 1 μm in dimension and the height thereof is at least 3 times the dimension of the base of the nanoshafts.

10. The chip of claim 1, wherein the nanoshafts formed on the interface region of the solid support are at a density greater than 10,000 nanoshafts/cm².

11. The chip of claim 1, which further comprises a device for applying the electric voltage or the electric current.

12. The chip of claim 1, which further comprises a carbon nanotube attached to the nanoshafts of the chip.

13. The chip of claim 1, which further comprises DNAs, RNAs, proteins, antibody, antigen immobilized onto the shafts or carbon nanotubes attached to the nanoshafts of the array.

14. The chip of claim 8, wherein the hollow nanoshafts further have a microflow channel therein along its length.

15. A method for delivering a sample to a target, which comprises the steps of coating the sample on the nanoshafts of the nanoneedle chip as defined in claim 1, and inserting the nanoshafts into the target, whereby the sample is delivered to the target.

16. A method for removing a sample from a target, which comprises the steps of inserting the nanoshafts of the nanoneedles chip as defined in claim 1, and taking out the chip, whereby the sample attached to the nanoshafts can be obtained.

17. The method of claim 15, wherein the target is selected from the group consisting of nuclei, mitochondria, chloroplasts, cells, tissues, organs, or removing samples from nuclei, mitochondria, chloroplasts, cells, tissues and organs.

18. The method of claim 16, wherein the target is selected from the group consisting of nuclei, mitochondria, chloroplasts, cells, tissues, organs, or removing samples from nuclei, mitochondria, chloroplasts, cells, tissues and organs.

19. A method for replicating DNA chip, which comprises the steps of immobilizing a strand of DNAs in a microwell plate, performing a polymerase chain reaction in the microwell, putting the nanoshafts of the nanoneedle chip as defined in claim 11 to the microwell plate, applying electric current to the nanoshafts so that the antisense strand of the DNAs can be bound to the nanoshafts, removing the nanoneedle chip to another microwell plate, and changing the polarity of the electric voltage whereby the antisense DNAs drop to the microwell plate.

20. A process for producing a solid nanoneedle chip, comprising the steps of:
(i) providing a solid support having an interface region;
(ii) coating a layer of a material on the interface region of the solid support to form nanoshafts wherein the material is selected from the group consisting of silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, ceramics and polymers;
(iii) coating photoresist on the layer of the material;
(iv) performing photolithography to form an array of dots;
(v) etching the material to transfer the dot patterns onto the solid support;
(vi) etching the solid support to a predetermined depth to define standing posts;
(vii) removing the photoresist and material on the standing posts; and
(viii) etching the posts by a chemical solution over a controlled time to form the cone-like or cylinder-shaped solid nanoshafts, wherein the top of each nanoshaft ranges from about 0.1 nm to less than about 1 μm in dimension, the base of each nanoshaft ranges from about 1 nm to less than about 1 μm in dimension, and the height of each nanoshaft is at least three times the dimension of the base.

21. The process of claim 20, wherein the solid support is selected from the group consisting of Si, GaAs, III-V semiconductor compounds, metals, ceramics, polymers, organic materials and glasses.

22. The process of claim 20, wherein the nanoshafts are made of the materials selected from the group consisting of wet silicon oxide, dry silicon oxide, chemical silicon oxide, silicon nitride, silicon carbide and silicide.

23. A process for producing a hollow nanoneedle chip, comprising the steps of:
(i) providing a solid support having an interface region;
(ii) coating a layer of a material to form nanoshafts on the interface region of the solid support, wherein the material is selected from the group consisting of silicon oxynitride, tetraethylorthosilicate, wet silicon oxide, dry silicon oxide, chemical silicon oxide, silicon nitride, silicon carbide, gallium arsenide, aluminum oxide, silicide, barium strontium titanate, lead zirconium tantalate, organic material, metals, metal oxides, conductors, ceramics and polymers;
(iii) coating photoresist on the layer of the material;
(iv) performing photolithography to form an array of dots;
(v) etching the material to transfer the dot patterns onto the solid support;
(vi) etching solid support to a predetermined depth to define the standing posts;
(vii) removing the photoresist and material on the posts;
(viii) growing a conformal film by atomic layer chemical vapor deposition, ultra high vacuum chemical vapor deposition, and displacement deposition to cover the whole solid support;
(ix) removing the upper layer of the conformal film by chemical mechanical polishing; and
(x) etching the solid support to form the hollow support and nanoshafts wherein the nanoshafts are in a shape of cone-like or cylinder and the top of each nanoshaft ranges from about 0.1 nm to less than about 1 μm in dimension, the base of each nanoshaft ranges from about 1 nm to less than about 1 μm in dimension, and the height of each nanoshaft is at least three times the dimension of the base.

24. The process of claim 23, wherein the solid support is selected from the group consisting of Si, GaAs, III-V semiconductor compounds, metals, ceramics and glasses.

25. The process of claim 23, wherein the nanoshafts are made of the materials selected from the group consisting of wet silicon oxide, dry silicon oxide, chemical silicon oxide, silicon nitride, silicon carbide and silicide.
